(19) United States
(12) Patent Application Publication
(10) Pub. No.: US 2010/0237281 A1
(43) Pub. Date: Sep. 23, 2010

(54) Peroxide Gel Compositions

(75) Inventor: Steven D. Jensen, South Jordan, UT (US)

Correspondence Address: CAO Group, Inc., 4628 W. Skyhawk Drive, West Jordan, UT, 84084, US

(73) Assignee: CAO GROUP, INC., West Jordan, UT (US)

(21) Appl. No.: 12/797,490

(22) Filed: Jun. 9, 2010

Related U.S. Application Data
(63) Continuation of application No. 11/307,463, filed on Feb. 8, 2006, now abandoned.

(51) Int. Cl. A61K 8/22; A61K 8/38; A61Q 11/00

(52) U.S. Cl. 252/186.42; 252/186.43

(57) ABSTRACT

The present invention is the use of Poly(2-ethyl-2-oxazoline) in the creation of peroxide gels for various applications. Such applications include bleaching of hair, teeth, laundry or any other bleachable item. Blending of the gel is accomplished by mixing the Poly(2-ethyl-2-oxazoline) with a peroxide such as hydrogen peroxide, carbamide peroxide, sodium perborate, or sodium percarbonate, usually also with water or an appropriate organic solvent. Peroxide concentrations in these new gels can reach a 30% concentration of hydrogen peroxide while maintaining a shelf life of six months at room temperature without developing peroxide decomposition.

### CROSS-REFERENCE TO RELATED APPLICATIONS

This patent application is a continuation of U.S. application Ser. No. 11/307,463, filed Feb. 8, 2006.

### FIELD OF THE INVENTION

The present invention relates to the field of thickeners and more particularly relates to a thickener for the development of a gel for the storage and delivery of peroxide, particularly hydrogen peroxide, for bleaching and other purposes.

### BACKGROUND OF THE INVENTION

Inorganic peroxide is usually defined as hydrogen peroxide and its adducts. Some examples are: hydrogen peroxide, carbamide peroxide, sodium percarbonate, sodium perborate. Peroxide is used in many different applications from an antiseptic for minor wounds to bleach for teeth, hair and laundry. Solutions of varying strengths of hydrogen peroxide are readily on the market, usually in a liquid form.

For targeted bleaching applications, such as tooth whitening, it is desirable to blend the peroxide into a gel carrier by blending the peroxide with a thickener. Blending is accomplished by mixing the thickener with the peroxide, usually also with water or an appropriate organic solvent. However, due to the volatile oxidizing nature of peroxide (which imparts the substance's bleaching ability); there are very few thickeners that can withstand a peroxide environment. Most polymers will degrade quickly in a peroxide environment and will lose their thickening properties entirely due to the powerful oxidizing effects of peroxide. These gels will degrade into thin, water-type consistencies. It is rare to find a polymer that can withstand, for prolonged periods of time, the powerful effects of peroxide.

Chemists have diluted hydrogen peroxide in order to tame its instability and raw oxidizing power. Liquid hydrogen peroxide is common and is by far the most aggressive oxidizer and the most unstable. Chemists have also produced adducts of hydrogen peroxide to stabilize the final concentration of hydrogen peroxide in the resultant compound. The main adducts of hydrogen peroxide that are used for bleaching are: urea hydrogen peroxide (carbamide peroxide), sodium perborate, and sodium percarbonate. However, dilution of hydrogen peroxide by any means, while increasing stability, also reduces the bleaching efficacy of resultant gels. In current use, the highest concentration of carbamide peroxide, which is 50% pure hydrogen peroxide by weight, in a dental bleaching gel is about 35%, yielding only 17.5% hydrogen peroxide. Sodium percarbonate has an even lower concentration of hydrogen peroxide, 30%. The use of these adducts then, generates an instant upper limit to the final concentration of hydrogen peroxide in a product.

Dental whitening manufacturers have predominately been using carbamide peroxide. Carbimide peroxide is docile enough to be used with many polymers that would not work with pure hydrogen peroxide. The most used commercial thickener, CARBOPOL, is a good example of this. CARBOPOL is good thickener for carbamide peroxide. However, CARBOPOL does not hold up to pure hydrogen peroxide for even short amounts of time. When CARBOPOL is used in a 30% strength hydrogen peroxide composition, the composition will begin to break down and form peroxide decomposition bubbles in about two weeks. Therefore what is needed is a polymer that is capable of withstanding hydrogen peroxide compositions for moderate amounts of time.

The present invention represents a departure from the prior art in that the application of the present invention in peroxide gels allows for higher peroxide concentrations by providing a gel base that is surprisingly stable in a peroxide environment. The resultant gels may use pure hydrogen peroxide at concentrations where only adducts have been used in the prior art, thereby doubling or tripling the resultant concentration of hydrogen peroxide in the finished product while simultaneously providing comparable or superior gel stability.

### SUMMARY OF THE INVENTION

In view of the foregoing disadvantages inherent in the known types of thickeners for peroxide gels, this invention provides an improved thickener. As such, the present invention's general purpose is to provide a new and improved thickener that is capable of maintaining a gel consistency for a peroxide gel while allowing for higher peroxide concentrations to increase efficacy.

Chemical solutions and gels containing hydrogen peroxide are well known in the art. In principle, the solutions and gels are made by combining a peroxide, solvents and a thickening agent. Varying degrees of viscosity and strength are easily generated by altering the base components' proportions and identities. For the purpose of this application, the preferred embodiment will be described as a dental whitening gel, though many other applications may be easily conceived and should be deemed to be included in this Application and its claims. Such additional applications include bleaching products for hair or laundry, where viscosity may not be as important as with a dental gel, but the principles and invention described herein, namely higher viscosity and bleaching strength, are equally applicable.

The novel thickening agent is Poly(2-ethyl-2-oxazoline). It is a polymer that swells upon absorption of liquids. Poly(2-ethyl-2-oxazoline) creates very viscous gels. There are many different molecular weights of Poly(2-ethyl-2-oxazoline) available commercially. These can be chosen to impart different physical properties to the gel for bleaching and other applications.

Poly(2-ethyl-2-oxazoline) is surprisingly a polymer that is capable of excellent compatibility with peroxide and imparts excellent thick viscous properties to the gel. Experience has shown that a 30% hydrogen peroxide gel made with Poly(2-ethyl-2-oxazoline) stays a gel during a six month storage at room temperature. Poly(2-ethyl-2-oxazoline) is a superior polymer in an oxidizing peroxide environment to current thickening polymers like CARBOPOL, silica, PVP, and polyethylene glycols.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims. Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### DETAILED DESCRIPTION OF THE INVENTION

The preferred embodiments of the peroxide gel compositions are herein described. It should be noted that the articles “a”, “an” and “the”, as used in this specification, include plural referents unless the content clearly dictates otherwise.

Poly(2-ethyl-2-oxazoline) is commercially available in 50,000, 200,000 and 500,000 M.W. Varying viscosities and longevity of gels may be created based on the amount and weight of Poly(2-ethyl-2-oxazoline) used and the desired strength of peroxide. As a guide, dental gels are preferred to be a viscosity between 1000 and 200,000 centipoise. In such ranges, peroxide concentrations may reach up to 50% hydrogen peroxide using Poly(2-ethyl-2-oxazoline) as a thickener. In its preferred form, a 30% concentration may be obtained with a shelf life of six months at room temperature. The simplest preferred gel is obtained by mixing 50% strength hydrogen peroxide with 200,000 M.W. Poly(2-ethyl-2-oxazoline) in a ratio of 6:4. Additional strengths of peroxide gels may be obtained by utilizing additional solvents and different molecular weights of Poly(2-ethyl-2-oxazoline). Some common solvents include: water, ethanol, polyethylene glycols, polypropylene glycols, glycerin, and propylene glycol. Any of these may be added for varying the consistency and properties of the gels created. However, each gel must be developed with the basic limitation that the strength of the peroxide in the gel makes the gel inherently more unstable

Although the present invention has been described with reference to preferred embodiments, numerous modifications and variations can be made and still the result will come within the scope of the invention. Such modifications include increasing or decreasing viscosity and peroxide concentration for various purposes. No limitation with respect to the specific embodiments disclosed herein is intended or should be inferred.

What is claimed is:

1. A dental tooth whitening and bleaching composition, comprising: a thickening agent consisting of poly (2-ethyl-2-oxazoline), a peroxide active and a solvent.

2. The dental tooth whitening and bleaching composition of claim 1, wherein the peroxide active is selected from the group consisting of: hydrogen peroxide, carbamide peroxide, sodium perborate and sodium percarbonate.

3. The dental tooth whitening and bleaching composition of claim 1, wherein the solvent is selected from the group consisting of: water, ethanol, polyethylene glycol, polypropylene glycol, glycerin and propylene glycol.

4. The dental tooth whitening and bleaching composition of claim 2, wherein the solvent is selected from the group consisting of: water, ethanol, polyethylene glycol, polypropylene glycol, glycerin and propylene glycol.

5. The dental tooth whitening and bleaching composition of claim 4, wherein the poly (2-ethyl-2-oxazoline) has a molecular weight in the range of 50,000 to 500,000.

6. The dental tooth whitening and bleaching composition of claim 5, wherein the composition has a viscosity in the range of 1,000 to 200,000 centipoise.

7. The dental tooth whitening and bleaching composition of claim 6, wherein the composition has a concentration of hydrogen peroxide in the range of about 30% to about 50% by weight.

8. The dental tooth whitening and bleaching composition of claim 7, wherein the composition has a concentration of hydrogen peroxide in the range of about 40% to about 50% by weight.

9. A dental tooth whitening and bleaching composition having a hydrogen peroxide concentration of at least 30% by weight and the ability to resist peroxide decomposition at room temperature for a period of up to six months, comprising: a thickening agent consisting of poly (2-ethyl-2-oxazoline), hydrogen peroxide and a solvent.

10. The dental tooth whitening and bleaching composition of claim 9, wherein the solvent is selected from the group consisting of: water, ethanol, polyethylene glycol, polypropylene glycol, glycerin and propylene glycol.

11. The dental tooth whitening and bleaching composition of claim 11, wherein the poly (2-ethyl-2-oxazoline) has a molecular weight in the range of 50,000 to 500,000.

12. The dental tooth whitening and bleaching composition of claim 12, wherein the composition has a viscosity in the range of 1,000 to 200,000 centipoise.

13. The dental tooth whitening and bleaching composition of claim 13, wherein the composition has a concentration of hydrogen peroxide in the range of about 30% to about 50% by weight.

14. The dental tooth whitening and bleaching composition of claim 14, wherein the composition has a concentration of hydrogen peroxide in the range of about 40% to about 50% by weight.

15. A dental tooth whitening and bleaching composition having a hydrogen peroxide concentration of at least 30% by weight and the ability to resist peroxide decomposition at room temperature for a period of up to six months, comprising: a thickening agent consisting essentially of poly (2-ethyl-2-oxazoline), hydrogen peroxide and a solvent.

16. The dental tooth whitening and bleaching composition of claim 15, wherein the solvent is selected from the group consisting of: water, ethanol, polyethylene glycol, polypropylene glycol, glycerin and propylene glycol.

17. The dental tooth whitening and bleaching composition of claim 16, wherein the poly (2-ethyl-2-oxazoline) has a molecular weight in the range of 50,000 to 500,000.

18. The dental tooth whitening and bleaching composition of claim 17, wherein the composition has a viscosity in the range of 1,000 to 200,000 centipoise.

19. The dental tooth whitening and bleaching composition of claim 18, wherein the composition has a concentration of hydrogen peroxide in the range of about 30% to about 50% by weight.

20. The dental tooth whitening and bleaching composition of claim 19, wherein the composition has a concentration of hydrogen peroxide in the range of about 40% to about 50% by weight.
